(19) United States
(12) Patent Application Publication
(10) Pub. No.: US 2011/0100369 A1
(43) Pub. Date: May 5, 2011

(54) Nasal Device for Obesity Prevention and Treatment

(76) Inventors: Liang Zhang, Alexandria, VA (US); Zhengping Zhang, Alexandria, VA (US)

(21) Appl. No.: 12/996,823

(22) PCT Filed: Apr. 6, 2009
(86) PCT No.: PCT/IB09/51443

§ 371 Date: Dec. 8, 2010

Related U.S. Application Data
(60) Provisional application No. 61/061,621, filed on Jun. 15, 2008.

(51) Int. Cl. A61M 16/00

(52) U.S. Cl. 128/206.11; 128/207.18

(57) ABSTRACT

A nasal device (10-16) for obesity prevention and treatment and its method of use are disclosed. The device comprises a check valve (20, 20′) and a cylindrical or conical airtight tube (30, 32′, 33, 34, 34′/36′) with an opening at each end. The valve is so fastened or connected to the tube at one end as to form a one-way air channel/passage. The tube conforms to or fits the interiors of different nostrils air-tightly in a variety of ways (32′, 34, 38, 40, 34′, 40′, 46). A pair of such devices can be connected with a flexible strip (42) or a band (44). Inserting the device into the nostril, a wearer can inhale through the nose but has to exhale through the mouth. This reduces the wearer's appetite for food and slows down eating. The wearer can easily remove the device after eating or whenever desired.

### TECHNICAL FIELD

This invention relates to medical devices for obesity prevention and treatment, specifically to a novel nasal device reducing its wearer's appetite for overeating.

### BACKGROUND ART

Obesity becomes a worldwide epidemic in our modern time, especially in developed countries; there is a strong association between development of obesity in adulthood and development of other serious diseases, including diabetes, hypertension, and heart disease. Therefore, desires for finding a simple, effective approach to its prevention and cure without the use of medication, thus without any side effects, are strong. Although many different diet methods using either food substitutes or willpower are created, none of them is actually effective in a long-term.

It is well known that our ‘taste’ of flavors actually strongly relies on our sense of smell. Anosmics, people who have lost their sense of smell due to illness or injury, often lose their appetite for food. Thus, a simple but impractical method to significantly reduce or even eliminate flavor perception temporarily is pinching the nose while chewing food because no air, thus no significant amount of food odors, flows from the mouth through the nasopharynx into the nasal cavity where olfactory receptors reside. So far, however, no medical devices have been developed to exploit such well-known phenomena in a practical way. In contrast, nasal filters have been developed over one century as filtering devices that users can either insert in the nostrils or cover the nose with.

U.S. Pat. No. 7,138,107 to Adams et al. discloses the use of medication—calcium channel blockers—to intentionally inhibit olfactory sensory perception to treat some health problems related to eating, including obesity. Given the olfactory bulbs' position inside the nasal cavity, any therapy based on the method would hardly be practical to individuals. In cases of rotten food or a fire emergency, such a therapy itself would be potentially harmful or dangerous since it intends to inhibit the sense of smell completely for a long time. Furthermore, such a therapy would result in involuntary abstinence, a serious side effect, because sex and smell may be linked in our brain by similar pathways (about a quarter of anosmics lose not only their appetite for food but also their interest in sex). Therefore, a safer, simpler, and more practical or effective approach is still needed.

Accordingly, an object of the present invention is to provide a simple device that assists the user to prevent or treat obesity. Another object of the invention is to provide a safe diet therapy that enables the dieter to use own biological system naturally and therefore has no perceivable side effects. Another object is to provide a long-term effective therapy for obesity prevention and treatment. Yet another object of the invention is to provide a therapy which effect on dieters can be on or off instantly as desired. Further objects and advantages of the present invention will become apparent after consideration of the following detailed descriptions and drawings.

### DISCLOSURE OF INVENTION

### SUMMARY OF INVENTION

This invention discloses a novel nasal device as a safe, simple, and convenient tool that helps the wearer diet. In accordance with several modes, the device basically comprises a check valve and a cylindrical or conical airtight tube with an opening at each end; the valve is so fastened or connected to the tube as to form a one-way channel or passage for air. The valve, preferably made of a polymer material such as polyethylene (PE) or polyvinyl chloride (PVC), allows airflow to pass through the tube from one end, but it stops airflow from the other. The tube can be made in a variety of ways or designs to conform to or fit the interiors of different nostrils air-tightly and comfortably. The device may be inserted into each nostril individually or in a pair, which two devices are connected with a flexible strip or band. The effects of the device can be turned on or off instantly as it is worn or removed. Before eating or after eating for certain time or amount, the user puts on the device to the nose. Then the wearer can breathe in through the nose but has to breathe out through the mouth. Because airflow is prevented from carrying chewed food odors to the olfactory receptors in the nasal cavity, the wearer loses flavor perception and, therefore, the appetite for food. Furthermore, the device does not allow the wearer to eat too fast, further reducing the chance of overeating. Meanwhile, the wearer retains the sense of smell for the environment. The device can be easily removed immediately after eating or whenever desired.

### DESCRIPTION OF DRAWINGS

FIG. 1 is a perspective sectional view of a nasal device with a one-way passage or channel that consists of a flap valve and two rings connected by a flexible airtight wall in accordance with one mode of the present invention.

FIG. 2 is a perspective sectional view of a nasal device with a one-way passage or channel that consists of a two-piece flexible valve and two rings connected by a flexible airtight wall in accordance with another mode of the present invention.

FIG. 3 is a perspective sectional view of a nasal device with a one-way passage or channel that consists of a flap valve and an elastic tube in accordance with another mode of the present invention.

FIG. 4 is a perspective view of a nasal device with a one-way passage or channel that consists of a two-piece flexible valve and an elastic ring in accordance with another mode of the present invention.

FIG. 5 is a perspective sectional view of a nasal device with a one-way passage or channel that consists of a flap valve and a tube coated with a layer of a soft, resilient, and airtight material in accordance with another mode of the present invention.

FIG. 6 is a perspective view of a nasal device with a one-way passage or channel that consists of a flap valve and a tube with two flexible flanges in accordance with another mode of the present invention.

FIG. 7 is a perspective view of a nasal device with a pair of single one-way passages or channels connected by a flexible strip in accordance with another mode of the present invention.

FIG. 8 is a perspective view of a nasal device with a pair of single one-way passages or channels fastened to a flexible adhesive band with two openings in accordance with another mode of the present invention.

FIG. 9 is an exploded view of a nasal device with a one-way passage or channel, a flexible flange, and a separable or removable check valve in accordance with another mode of the present invention.

### MODES FOR INVENTION

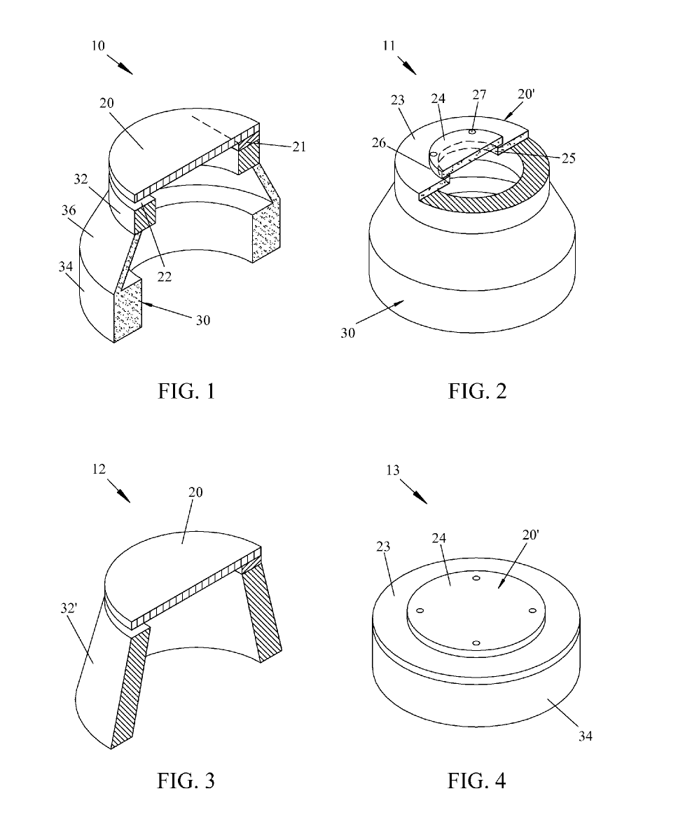

A mode of the present invention, a device 10, is illustrated in FIG. 1. The device comprises a flap or valve 20 and a substantially cylindrical or conical tube, passage, or channel 30. The channel consists of a tube or ring 32 preferably made of a substantially rigid or semi-rigid material, a tube or ring 34 preferably made of a substantially elastic material such as an elastomer, and a flexible conical tube or wall 36 opened at both ends and made of a sheet of a flexible airtight material such as flexible polyvinyl chloride (PVC). Ring 34 is preferably larger in diameter than ring 32 and can readily change its shape to conform to or engage with external forces. Wall 36 connects both rings air-tightly with its open-ends to only allow air to flow through the rings. Furthermore, ring 32 does not change its shape when ring 34 is compressed, stretched, or deformed. Valve 20 consists of a thin disc or a piece of film made of a light, flexible, and substantially rigid airtight material and is connected to the top surface of ring 32 to form a standard check valve, as well-known in the art, with a hinge 21 and a fine gap or space 22 in its normal or relaxed position. Being operable by airflow, the check valve stops airflow from its side, but it allows airflow from the ring's side to pass through channel 30.

Another mode of the present invention, a device 11, is illustrated in FIG. 2. The device is almost identical to device 10 except that valve 20 is replaced by a check valve 20′ that consists of two thin discs, discs 23 and 24, each made of a sheet of a flexible airtight material such as vinyl. Disc 23 has a hole or opening 25 (as shown by dashed circle) at its center, is connected to ring 32 air-tightly on one side, and contacts with disc 24 closely on the other side. Disc 24 is preferably smaller than disc 23, but large enough to cover opening 25, and is connected or fastened onto disc 23 at a plurality of spaced points 27 about the perimeter of opening 25. Valve 20′ only allows airflow from channel 30 to pass through space 26 between points 27, as known in the art. Under airflow pressure from its side, disc 24 lies flat against disc 23, thereby sealing opening 25 and stopping the airflow; while under airflow pressure from the other side, disc 24 is pushed away from disc 23 so that the airflow passes opening 25 and space 26 between the connection points.

Another mode of the present invention, a device 12, is illustrated in FIG. 3. The device is identical to device 10 except that channel 30 is replaced by conical tube or channel 32′, preferably made of an elastic material. The tube can also be viewed as an expansion of ring 32 to engage with the nostril; its size and shape are predetermined, preferably according to those of typical nostrils. Similar to device 10, airflow through tube 32′ can pass valve 20 but cannot pass the valve from the other side to enter the tube.

Another mode of the present invention, a device 13, is illustrated in FIG. 4. The device is formed by connecting valve 20′ to ring 34 the same way as it is connected to ring 32, as described for device 11.

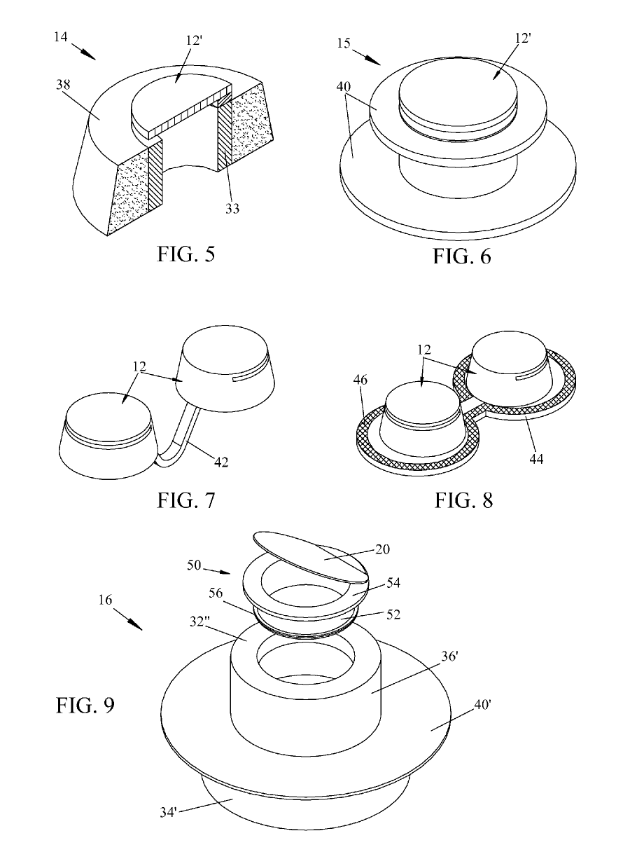

Another mode of the present invention, a device 14, is illustrated in FIG. 5. The device consists of a device 12′, which is similar to device 12 but has a tube 33 made of a rigid or semi-rigid material, and a layer 38 of an elastic, soft, and airtight material such as silicone. Device 12′ is coated with the layer on its outer surface to improve its conformability. An alternative way to improve the conformability of device 12′ is illustrated in FIG. 6 for a device 15, another mode of the present invention. Two flexible flanges 40 extend radially outwardly from the outer wall of tube 33 at both ends. The flanges are quite thin; their shapes and relative positions to the axis of the tube can be varied. Both layer 38 and flange 40 enables device 12′ to readily conform to the nostril despite variations of nostrils in size.

Another mode of the present invention, a device 16, is illustrated in FIG. 9. The most significant feature of the device, different from others as described above, is its check valve 50 that is separable or removable from its main body—an air channel. The channel consists of a conical tube 34′ with a radically outward flange 40′ and a cylindrical tube 36′ with a radically inward flange 32′. The channel is preferably molded in one piece with a resilient material such as a soft elastomer; therefore, it can conform to the interior walls of the nostril. The resilience of tube 34′ is gentle but strong enough to retain the device inside the nostril. Tube 36′ is thinner, smaller in diameter, and therefore more flexible than tube 34′, preferably avoiding contact with the nostril. These two tubes are connected air-tightly, similar to the configuration of ring 32, ring 34, and wall 36 in device 10. Removable valve 50, preferably made of a rigid or semi-rigid material, has a ring 52 with a radially outward flange 54 at one end and optionally an annular snap rim 56 at the other, in addition to flap 20. The outer diameter and the height of ring 52 are chosen to be slightly larger than the inner diameter and the thickness of flange 32′, respectively. Therefore, the removable valve can be snapped in and secured with flange 32′ air-tightly.

The usage or operation of devices 10-16 is generally described as follows. Before eating or after eating for certain time or amount, the user inserts such a device into the nostril, with the valve end first, and pushes the other end into the nostril with a fingertip until the device resides inside the nostril. The device conforms to the nostril and thereby seals the nostril air-tightly. It can fit different types of noses, such as the Caucasian, Oriental, or Negroid one, or different sizes through the deformation or conformation of ring 34, tube 32′, layer 38, flange 40, or tube 34′. Thus, airflow can only enter into the nose through the device. Furthermore, their elasticity or resilience helps to retain the device in place and prevent inadvertent removal of the device from the nostril. Such conformation does not prevent check valve 20 or 20′ from function properly because of either the flexibility of valves 20, 20′ or the rigidity of ring 32, tube 33, or tube 52. The major function of flexible flange 40′ of device 16 is to further prevent air from leaking between the exterior of tube 34′ and the interior walls of the nostril. During eating, the wearer can breathe in through the nose but has to breathe out through the mouth. Therefore, similar to pinching the nose while chewing, no air carrying significant amount of chewed food odors flows from the mouth through the nasopharynx into the nasal cavity where olfactory receptors reside. As a result, the wearer loses significantly or completely the flavor perception and, therefore, the appetite for food. Furthermore, because of the time needed for the exhalation through the mouth, the wearer cannot eat too fast, further avoiding unconsciously overeating. However, wearing the device does not disturb wearer's sense of smell through the nose for sensing potential unhealthy or even dangerous environment or situation. To stop the effects of the devices, the wearer can remove them by blowing while closing the mouth or by pulling.

A pair of devices 10-16 can be connected with a flexible strip 42 at the open-ends of the tubes, as shown for a pair of devices 12 in FIG. 7. Although the strip is visible when wearing, it makes the removal of the pair much easier. As illustrated in FIG. 8, such a pair can also be fastened onto a support sheet or band 44 made of a flexible airtight material with two openings enclosed by the devices and separated by the distance between two nostrils. Each device is fastened or connected air-tightly, by its non-valve end, to the periphery or perimeter of each opening of band 44, respectively, on one side. Furthermore, an adhesive material or substance 46 is disposed continuously at borders of the band on the same side to form a specially shaped adhesive strip used for airtight attachment to the exterior of the nose. The user inserts the devices into the nostrils and fastens the adhesive strip to the exterior of the nose. The adhesive strip can hold the devices in place and seal the air passages between the devices' exteriors and the nostrils' interiors. One advantage of band 44 is to make the device's airtight fit to the nostril unnecessary. Alternatively, a pair of valves 20′ or only discs 24 can be directly fastened or connected to band 44, in place of the devices, to control airflow through the openings (not shown). After use, the devices can be easily removed.

### INDUSTRIAL APPLICABILITY

A number of advantages of the present invention, from the description above, are evident: it is safe, simple, discreet, easy to use, cheap to make, and useable whenever desired. Its use results in no perceivable side effects. The device just enables the wearer to use own biological system naturally. It is also effective by helping the wearer to control the food intake and never to overeat for the lifetime. Its effect is on or off instantly whenever it is worn or removed as desired.

While the above description contains many specificities, these should not be construed as limitations on the scope of the invention, but as exemplifications of the presently preferred modes thereof. Many other ramifications and variations are possible within the teachings of the inventions.

For example, in general, the approach is also applicable to other mammals, such as pets. There are also two extreme cases, not included in the descriptions above: 1) a nasal plug that completely blocks the nostril; and 2) a nasal filter that reduces, not completely but significantly, airflow from mouth to nose. Forcing both the inhalation and the exhalation through the mouth, the former can also reduce the appetites although the method is unhealthy. The latter, such as nasal filters with very fine filtering capability, may significantly weaken the exhalation airflow through the nose and therefore reduce wearers' appetites in certain degree.

Furthermore, the device may alternatively or additionally comprise the following: 1) a check valve with a different design or mechanism from the modes described; 2) a tube with a different conformation structure or mechanism, such as tubes with a plurality of flanges greater than two or with a soft coat on the exterior in multiple pieces or one piece with multiple slits; 3) a tube made in a variety of shapes, sizes, materials, and textures, such as an elliptical tube; 4) a different configuration from what are illustrated in the modes described, such as a valve installed in the middle, instead of the end, of the tube; 5) smoothed or rounded edges of the tubes for comfort or to guide insertion; 6) an adhesive band with a different shape or with complete or partial coverage of adhesive materials; 7) a check valve controlling one opening of an adhesive band for a single nostril; and 8) different combinations of the modes and alternatives described above, such as a one-way passage with a filter or filtering material.

Thus the scope of the invention should be determined not by the modes illustrated, but by the appended claims and their legal equivalents.

1. A nasal device, wearable for obesity prevention and treatment, comprising: an air passageway, a wear means for securing said device to a nose, and a valve means for controlling airflow so that air can pass through said passageway only or substantially in one direction, whereby the wearer can inhale but cannot exhale through said nose.

2. The nasal device of claim 1 wherein said air passageway is partially or completely formed by a channel or tube made of at least one airtight material.

3. The nasal device of claim 2 wherein said channel or tube includes a seal means for sealing air passages, if any, between the exterior thereof and the nostril substantially air-tightly.

4. The nasal device of claim 2 wherein said wear means includes an elastic ring or tube that is conformable to the nostril so that said device can be secured inside said nostril.

5. The nasal device of claim 2 wherein said wear means includes at least one flexible flange that is conformable to the nostril so that said device can be secured inside said nostril.

6. The nasal device of claim 3 wherein said seal means consists of a layer of a soft, resilient, or airtight material coating said exterior partially or completely, whereby said nasal device can fit or seal different nostrils by the conformation of said layer.

7. The nasal device of claim 3 wherein said seal means includes at least one flexible flange made of a sheet of a flexible airtight material, whereby said nasal device can fit or seal different nostrils by the conformation of said flexible flange or flanges.

8. The nasal device of claim 2 wherein said valve means is separable from said channel or tube.

9. The nasal device of claim 2 wherein said valve means includes a check valve or the like that is operable by airflow.

10. The nasal device of claim 9 wherein said check valve includes a flap that is made of a sheet or film of a light, airtight material.

11. The nasal device of claim 3 wherein said channel includes a flexible band with a hole or opening; said wear means includes an adhesive strip that is formed with an adhesive material disposed on said band.

12. The nasal device of claim 11 wherein said seal means includes said adhesive strip surrounding or enclosing said hole or opening.

13. The nasal device of claim 11 wherein said valve means includes a flexible disc or flap that is connected or fastened to said band and can cover up said hole or opening on the same side as said adhesive strip.

14. The nasal device of claim 2 wherein said channel or tube includes a filtering material or filter that is secured therein.

15. The nasal device of claim 1 wherein said wearer can be either a human or an animal.

16. A nasal device, wearable for obesity prevention and treatment, comprising: a pair of air channels or tubes opened at both ends and two airflow-operable check valves or the like connected permanently or detachably to said pair respectively so as to allow air to pass through said pair only or substantially in one direction, whereby the wearer—either a human being or an animal—can breathe in through the nose but has to breathe out through the mouth.

17. The nasal device of claim 16 wherein each of said pair includes a flexible flange that is made of a sheet of a flexible, airtight material, is fastened to, connected to, or integrated with each exterior of said pair air-tightly, and extends outwardly from said each exterior substantially radially, whereby said flexible flange can seal air passages, if any, between the nostrils' interior walls and said each exterior substantially air-tightly when said device is worn.

18. The nasal device of claim 16 wherein each exterior of said pair is coated with a layer of a soft, resilient, and/or airtight material partially or completely, whereby said layer can seal air passages, if any, between each nostril's interior walls and said each exterior when said device is worn.

19. The nasal device of claim 16 wherein said pair are connected by a flexible strip or band or the like.

20. The nasal device of claim 16 wherein said pair of air channels includes two holes or openings, separated by the distance between the nostrils and sealed by said check valves, in a flexible sheet or band with at least one closed strip of an adhesive material disposed continuously around said holes or openings.

21. The nasal device of claim 16 wherein each of said pair includes a frustum-shaped flexible tube that is connected air-tightly at the smaller end to each of said check valves respectively and at the larger end to an elastic ring or tube; and said smaller end is preferably reinforced, whereby said check valves can function properly even when said elastic ring or tube is distorted.

22. The nasal device of claim 16 wherein each of said check valves includes first and second discs, each made of a sheet or film of a flexible airtight material; said first disc has a hole or opening and is connected or fastened respectively to each of said pair air-tightly; and said second disc covers up said hole or opening and is fastened to said first disc at a plurality of spaced points about the perimeter of said hole or opening.

23.-28. (canceled)
